(19) United States
(12) Patent Application Publication
(10) Pub. No.: US 2007/0020744 A1
(43) Pub. Date: Jan. 25, 2007

(54) Enzyme compositions that enhance the flavor of food and beverages

(75) Inventors: James F. Jolly, Elgin, IL (US); Sandra M. Ulbrich, Elgin, IL (US)

Correspondence Address: FOLEY AND LARDNER LLP;SUITE 500, 3000 K STREET NW, WASHINGTON, DC, 20007, US

(73) Assignees: Amano Enzyme USA Co., Ltd; Amano Enzyme, Inc.

(21) Appl. No.: 11/486,423

(22) Filed: Jul. 14, 2006

Related U.S. Application Data
(60) Provisional application No. 60/699,368, filed on Jul. 15, 2005.
(60) Provisional application No. 60/815,837, filed on Jun. 23, 2006.

(51) Int. Cl. C12N 9/00

(52) U.S. Cl. 435/183

(57) ABSTRACT

The invention provides enzyme compositions that are useful for enhancing the nutritional value and/or flavor of food and beverage products. The invention also provides a process for producing such food and beverage products that have enhanced nutritional and/or flavor profiles.

### CROSS-REFERENCE TO RELATED APPLICATIONS

This application claims the benefit under 35 U.S.C. § 119(e) to U.S. Provisional Application No. 60/699,368, filed on Jul. 15, 2005, and Provisional Application No. 60/815,837 filed on Jun. 23, 2006 which are incorporated herein by reference in their entirety.

### BACKGROUND OF THE INVENTION

The following discussion of the background of the invention is merely provided to aid the reader in understanding the invention and is not admitted to describe or constitute prior art to the invention.

A glycone (also referred to as a “glycoside”) is a molecule that is conveniently viewed as a saccharide bonded to a non-saccharide moiety called an aglycone (also referred to as “aglycons”). Glycones are prevalent in nature, and many are associated with physiological benefits.

For example, the subset of glycones known as isoflavone glycones (IFG) contain isoflavones as the aglycones. Some isoflavones have been shown to have antioxidant activity, and act to protect the cells from the damaging effects of free radicals such as reactive oxygen species (e.g., singlet oxygen), superoxide, hydroxyl radicals, etc. High levels of reactive oxygen species have been show to lead to oxidative stress, which has been linked to diseases such as Parkinson's and Alzheimer's, cardiovascular diseases such as atherosclerosis, and the exacerbation of some types of cancers. Accordingly, many isoflavone aglycones are associated with the prevention and symptom-alleviating effects of diseases such as cancer, arteriosclerosis, osteoporosis, climacteric disorder, diseases related to aging, and lowering blood cholesterol.

Usually, glycones do not themselves exhibit physiological activity, but rather such activity has been attributed to the aglycones resulting from enzymatic hydrolysis (e.g., sugar cleavage) of the glycones. Thus, it may be necessary to liberate the algycones to realize the physiological benefits. Moreover, free aglycone, such as isoflavones and flavonoids give rise to increased bioavailability, faster absorption, higher efficiency, and stronger bioactivity relative to the corresponding glycones that are naturally hydrolyzed in physiological processes.

Many food and beverages contain glycones that will yield aglycones giving rise to the physiological benefits mentioned above. Moreover, the liberation of some aglycones result in flavor enhancement, i.e., the enhancement of pleasing aromas, the enhancement of taste, or both. This additional benefit is of great commercial importance in the manufacture of food products and beverages that contain the requisite glycones. Enhancing the flavor of such products can reduce or mask unpleasant aspects of the product, strengthen desirable tastes and aromas, or both. Additionally, the liberation of aglycones associated with advantageous physiological effects offers further benefits, such as improved nutritional, nutraceutical or therapeutic value. This may be particularly beneficial for populations or specific subjects (including human or animal subjects) that do not effectively or efficiently hydrolyze glycones into their beneficial aglycone forms (e.g., whose digestive systems lack the enzyme activity needed to effectively or efficiently hydrolyze the beneficial glycone).

Techniques are known in the art for converting glycones into aglycones. A conventional technique implicates enzymatic cleavage of the glycone to yield aglycones. Specifically, glucosidase will achieve the desired conversion, but this process suffers from a number of drawbacks. It requires multiple steps, often gives rise to harmful by-products and further degradation, and is an overall inefficient chemical process. It would therefore be desirable to liberate aglycones in a single efficient step.

Additionally, foods and beverages do not all present similar glycone profiles. Indeed, contacting a food or beverage with only one enzyme such as glucosidase may not realize the full potential of flavor enhancing aglycones that are available. Thus glycosidic cleavage may result in the release or partial release of some aglycones, leaving yet other aglycones unreleased. Therefore it would be desirable to enhance the flavor of foods and beverages with broad glycone profiles in one convenient step.

### SUMMARY OF THE INVENTION

The present invention satisfies these needs and others by providing, in some embodiments, food or beverage compositions comprising or treated with an enzyme composition, and methods of preparing such food or beverage compositions.

In accordance with one aspect, the invention provides a food or beverage composition comprising (i) a food or beverage comprising a glycone and (ii) an enzyme composition exhibiting an enzyme activity profile that includes one or more of glucosidase activity, β-glycosidase activity, protease activity, lipase activity, amylase activity, glucoamylase activity, xylanase activity, and pectinase activity. In specific embodiments, the food or beverage composition exhibits an increased aglycone content and/or enhanced flavor relative to a corresponding composition that does not comprise the enzyme composition.

In one embodiment, the enzyme activity profile of the enzyme composition includes one or more of a glucosidase activity of about 40 to about 70 u/g; a β-glycosidase activity of about 0.3 to about 0.9 u/g; a protease activity of about 4,000 to about 8,000 u/g; a lipase activity of about 300 to about 500 u/g; an amylase activity of about 160,000 to about 190,000 u/g; a glucoamylase activity of about 24,000 to about 28,000 u/g; a xylanase activity of about 11,000 to about 14,000 u/g, and a pectinase activity of about 40 to about 120 u/g. In one embodiment, the enzyme composition comprises one or more enzymes selected from the group consisting of glucosidase, β-glycosidase, protease, lipase, amylase, glucoamylase, xylanase, and pectinase. In another embodiment, the enzyme composition comprises Protease M.

In accordance with another aspect, the invention provides a food or beverage composition prepared by a process comprising contacting a food or beverage comprising a glycone with an enzyme composition exhibiting an enzyme activity profile that comprises one or more of glucosidase activity, β-glycosidase activity, protease activity, lipase activity, amylase activity, glucoamylase activity, xylanase activity, and pectinase activity. In specific embodiments, the food or beverage composition exhibits an increased aglycone content and/or enhanced flavor relative to a corresponding composition that does not comprise the enzyme composition. In one embodiment, the enzyme composition comprises one or more enzymes selected from the group consisting of glucosidase, β-glycosidase, protease, lipase, amylase, glucoamylase, xylanase, and pectinase. In another embodiment, the enzyme composition comprises Protease M.

In accordance with another aspect, the invention provides a method of enhancing the flavor of a food or beverage comprising contacting a food or beverage with an enzyme composition exhibiting an enzyme activity profile that comprising one or more of glucosidase activity, β-glycosidase activity, protease activity, lipase activity, amylase activity, glucoamylase activity, xylanase activity, and pectinase activity. In one embodiment, the enzyme composition comprises one or more enzymes selected from the group consisting of glucosidase, β-glycosidase, protease, lipase, amylase, glucoamylase, xylanase, and pectinase. In another embodiment, the enzyme composition comprises Protease M.

In accordance with another aspect, the invention provides a method of increasing the aglycone content of a food or beverage comprising contacting a food or beverage comprising a glycone with an enzyme composition exhibiting an enzyme activity profile that comprises one or more of glucosidase activity, β-glycosidase activity, protease activity, lipase activity, amylase activity, glucoamylase activity, xylanase activity, and pectinase activity. In one embodiment, the enzyme composition comprises one or more enzymes selected from the group consisting of glucosidase, β-glycosidase, protease, lipase, amylase, glucoamylase, xylanase, and pectinase. In another embodiment, the enzyme composition comprises Protease M.

In accordance with another aspect, the invention provides an enzyme composition comprising one or more of glutaminase, β-glycosidase, protease, lipase, amylase, glucoamylase, xylanase, and pectinase. In one specific embodiment, the composition further comprises one or more of enzyme RP-1, deaminase and glutaminase. In another specific embodiment, the composition comprises an enzyme activity profile comprising one or more of a β-glycosidase activity of about 0.3 to about 0.9 u/g; a protease activity of about 4,000 to about 8,000 u/g; a lipase activity of about 300 to about 500 u/g; an amylase activity of about 160,000 to about 190,000 u/g; a glucoamylase activity of about 24,000 to about 28,000 u/g; a xylanase activity of about 11,000 to about 14,000 u/g, and a pectinase activity of about 40 to about 120 u/g.

In accordance with another aspect, the invention provides a food or beverage product, wherein the product comprises a flavor-enhancing amount of an enzyme composition having an enzyme activity profile comprising one or more of β-glycosidase activity, protease activity, lipase activity, amylase activity, glucoamylase activity, xylanase activity, and pectinase activity. In one specific embodiment, the enzyme activity profile further comprises glutaminase activity. In one embodiment, the enzyme composition comprises one or more enzymes selected from the group consisting of β-glycosidase, protease, lipase, amylase, glucoamylase, xylanase, and pectinase. In another embodiment, the enzyme composition comprises Protease M.

In accordance with another aspect, the invention provides a process for producing a food or beverage product having an enhanced flavor profile, comprising the step of contacting the food or beverage product with a flavor-enhancing amount of an enzyme composition having an enzyme activity profile comprising one or more of β-glycosidase activity, protease activity, lipase activity, amylase activity, glucoamylase activity, xylanase activity, and pectinase activity, whereby the flavor profile of the food or beverage product is enhanced. In one embodiment, the enzyme composition comprises one or more enzymes selected from the group consisting of β-glycosidase, protease, lipase, amylase, glucoamylase, xylanase, and pectinase. In another embodiment, the enzyme composition comprises Protease M. In one specific embodiment, the process further comprises, after the contacting step, the step of heating the food or beverage product for a time and at a temperature sufficient to inactivate said enzyme composition. The invention also provides a food or beverage product obtained by this process.

### BRIEF DESCRIPTION OF THE DRAWINGS

FIG. 1 shows a chromatograph of high performance liquid chromatography (“HPLC”) analysis of cranberry juice. The left panel shows glycone peaks before acid hydrolysis; the right panel shows glycone peaks decreasing as a result of acid hydrolysis.

FIG. 2 shows a chromatograph of high performance liquid chromatography analysis of cranberry juice. The left panel shows aglycone peaks before acid hydrolysis; the right panel shows aglycone peaks increasing as a result of acid hydrolysis.

FIG. 3 shows a chromatograph of a high performance liquid chromatography analysis of cranberry juice before (left panel) and after (right panel) treatment with Protease M.

FIG. 4 shows a chromatograph of a high performance liquid chromatography analysis of cranberry juice before (left panel) and after (right panel) treatment with β-glycosidase.

FIG. 5 shows an evaluation of color loss from and taste of 100% cranberry juice that was treated with varying doses of Protease M (“●” dark circle) or β-glycosidase (“▪”) as compared to untreated juice (control; “●” light circle). Taste characteristics at each enzyme or enzyme mixture dose are presented relative to untreated juice as being either improved taste or altered taste.

FIG. 6a-6d show chromatographs of a high performance liquid chromatography analysis of grape juice before (left panel) and after (right panel) acid hydrolysis (a and b); β-glycosidase treatment (c); and Protease M treatment (d).

FIG. 7a-7d show chromatographs of a high performance liquid chromatography analysis of cherry juice before (left panel) and after (right panel) acid hydrolysis (a and b); β-glycosidase treatment (c); and Protease M treatment (d).

FIG. 8a-8d show chromatographs of a high performance liquid chromatography analysis of blueberry juice before (left panel) and after (right panel) acid hydrolysis (a and b); β-glycosidase treatment (c); and Protease M treatment (d).

FIG. 9 shows an evaluation of color loss from and taste of 100% cranberry juice that was treated with varying doses of an enzyme composition comprising Protease M (“●” dark circle) and β-glycosidase (“▪”) as compared to untreated juice (control; “●” light circle). Taste characteristics at each enzyme or enzyme mixture dose are presented relative to untreated juice as being either enhanced (“+”) or altered (“−”). The data was further analyzed for statistical significance of colored precipitation, where “*” indicates P<0.001 compared to the control, and “**” indicates P<0.001 for the values of β-glycosidase compared to those for the enzyme composition.

FIG. 10 shows an evaluation of color loss from and taste of a cranberry and apple juice mixture that was treated with varying doses of an enzyme composition comprising Protease M (“●” dark circle) and β-glycosidase (“▪”) as compared to untreated juice (control; “●” light circle). The symbols “+”, “−”, “*”, and “**” have the same meanings as defined above for FIG. 10.

FIG. 11 shows an evaluation of color loss from and taste of a cranberry and tea mixture that was treated with varying doses of an enzyme composition comprising Protease M (“●” dark circle) and β-glycosidase (“▪”) as compared to untreated juice (control; “●” light circle). The symbols “+”, “−”, “*”, and “**” have the same meanings as defined above for FIG. 10.

FIG. 12 shows an evaluation of color loss from and taste of grape juice that was treated with varying doses of an enzyme composition comprising Protease M (“●” dark circle) and β-glycosidase (“▪”) as compared to untreated juice (control; “●” light circle). The symbols “+”, “−”, “*”, and “**” have the same meanings as defined above for FIG. 10.

### DETAILED DESCRIPTION

The invention provides enzyme compositions useful for enhancing the nutritional value and/or flavor (e.g., the taste and/or aroma), of foods and beverages. In accordance with one embodiment, a food or beverage comprising or treated with an enzyme composition exhibits enhanced flavor as compared to a corresponding untreated food or beverage. In accordance with another embodiment, a food or beverage comprising or treated with an enzyme composition exhibits an increased aglycone content as compared to a corresponding untreated food or beverage.

The present invention is described herein using several definitions, as set forth below and throughout the application.

As used herein, unless otherwise stated, the singular forms “a,” “an,” and “the” includes plural reference.

As used herein, the term “aglycone” (also known as “aglycon”) refers to a compound that is obtained from a glycone (also known as a “glycoside”) by the formal removal of a saccharide from the glycoside. Aglycones of glycones are ubiquitous in nature. Examples of aglycones include but are not limited to volatile compounds in plants such as linalool, geraniol, citronellal, phenethyl alcohol, citronellol, jasmones, limonene, terpinene, citral, nerol, pinene, bomeol, terpineol, methyl jasmonate, hexanol, hexenol, hexanal, hexenal, vanillin, benzaldehyde, eugenol, methyl salicylate, linalool oxide, benzyl alcohol, and vomifomitol; pigments in plants such as alizarin, purpurin, anthocyanidin including pellagonidin, cyanidin, delphinidin, peonidin, petunidin, and malvidin; and flavonoids such as nariltin, naringenin, hesperetin, neohesperetin, diosmetin, quercetin, campherol, myricetin, isorhamnetin, and syringenin; and the like. Other than the compounds mentioned herein, various compounds may be present as aglycones of glycones or may become aglycones of glycones.

The term “food product,” “food,” and “beverage product” or “beverage” as used herein refers not only to basic food or beverage ingredients but also to semi- and fully processed products that comprise one or more basic ingredients. Exemplary food products therefore include, but are not limited to, products consisting of or comprising meats, dairy products, oils, sweeteners, legumes, vegetables and vegetable products, fruits and fruit products, seasonings, grains, nuts and seed products, soy and soy products, and combinations thereof. Exemplary beverage products include coffee, tea, milk- and cream-based beverages, alcoholic beverages such as wines and beers, fruit and vegetable juices, for example, apple juice, cherry juice, pomegranate juice, grape juice, cranberry juices, citrus juices such as lemon juice (e.g., lemonade), orange juice, grapefruit juice, and mixtures of any of these beverages.

I. Enzyme Compositions

The present invention provides compositions having enzyme activity profiles comprising certain combinations of enzyme activities (including compositions comprising mixtures of enzymes and compositions obtained from enzyme-producing organisms that exhibit a plurality of enzyme activities) useful for enhancing the nutritional value and/or flavor (e.g., taste and/or aroma) profiles of a wide variety of foods and beverages, that is not possible or is less practical to obtain by the separate applications of individual enzymes.

As used herein, the phrase “enzyme activity profile” refers to the enzyme activities exhibited by a given enzyme composition. By “enzyme activity” is meant the activity of the named enzyme. Thus, for example, a composition having an enzyme activity profile comprising glutaminase activity exhibits the activity of glutaminase. As described herein, an enzyme composition may exhibit a plurality of different enzyme activities, which activities can be determined by routine assays well-known in the art, and discussed below.

The enzyme activities of an enzyme composition of the present invention may vary depending on the food or beverage to be treated and the desired characteristics of the treated product. For example, in some embodiments, the enzyme composition may comprise one or more of the following enzymes (or may exhibit the activity of one or more of the following enzymes): glutaminase, β-glycosidase, protease, lipase, amylase, glucoamylase, xylanase, pectinase, 5′ ribonuclease (RP-I) and deaminase. Examples of specific compositions of the above named enzymes include but are not limited to compositions comprising β-glycosidase, protease, lipase, amylase, glucoamylase, xylanase, and pectinase; compositions comprising glutaminase and β-glycosidase; compositions comprising glutaminase, β-glycosidase, protease, lipase, amylase, glucoamylase, xylanase, and pectinase; compositions comprising glutaminase, β-glycosidase, and enzyme RP-1; compositions comprising glutaminase, β-glycosidase, and deaminase; compositions comprising glutaminase, β-glycosidase, deaminase, and enzyme RP-1; and compositions comprising RP-1 alone (or as the sole enzyme component).

In other embodiments, the enzyme composition may exhibit an enzyme activity profile that includes one or more of, for example, glucosidase activity, β-glycosidase activity, protease activity, lipase activity, amylase activity, glucoamylase activity, xylanase activity, and pectinase activity. In one specific embodiment, the enzyme composition may comprise Protease M.

The following description of enzymes and enzyme activity is meant to aid the reader in understanding particular embodiments; it is not, however, meant to be limiting of the scope of the invention.

Glutaminase:

As mentioned above, some compositions may comprise glutaminase or glutaminase activity. Glutaminase can convert glutamine to glutamic acid, which is a well known flavor enhancer.

Protease:

Some compositions may include a protease or protease activity. Proteases are enzymes that break peptide bonds between the amino acids of proteins. There are currently six classes of proteases: serine proteases, threonine proteases, cysteine proteases, aspartic acid proteases (e.g. plasmepsin), metalloproteases, and glutamic acid proteases.

Lipase:

Some compositions may include a lipase or lipase activity. A lipase is a water-soluble enzyme that catalyzes the hydrolysis of ester bonds in water-insoluble, lipid substrates. Most lipases act at a specific position on the glycerol backbone of a lipid substrate.

Amylase:

Some compositions may include an amylase or amylase activity. Amylase is a digestive enzyme classified as a saccharidase, an enzyme that can cleave polysaccharides.

Glucoamylase:

Some compositions may include glucoamylase or glucoamylase activity. Glucoamylase (also known as amyloglucosidase) is an enzyme that breaks down glucose polymer structures. Glucoamylase is used in industrial saccharification steps, both in starch enzymatic conversion and in alcohol production.

Xylanase:

Some compositions may include xylanase or xylanase activity. Xylanase degrades the linear polysaccharide beta-1,4-xylan into xylose, thus breaking down hemicellulose, which is a major component of the cell wall of plants.

Pectinase:

Some compositions may include pectinase or pectinase activity. Pectinase is a general term for enzymes that break down pectin, a polysaccharide substrate that is found in the cell walls of plants. One of the most studied and widely used commercial pectinases is polygalacturonase.

RP-1 and Deaminase:

Other embodiments may provide compositions that comprise one or more additional enzymes such as RP-1 and deaminase. RP-1 degrades RNA to CMP, UMP, AMP, and GMP. Deaminase converts AMP to IMP. It should be recognized that GMP and IMP are flavor enhancers. Thus, in some embodiments, a composition according to this invention comprises both RP-1 and deaminase. In other embodiments, the composition comprises RP-1 and not deaminase.

Glucosidase:

Some compositions may include glucosidase, or glucosidase activity. Glucosidases are characterized as enzymes which catalyze the hydrolysis of glucosides (a glycone, the sugar component of which is glucose).

β-glycosidase:

Some compositions may include β-glycosidase, or β-glycosidase activity. β-glycosidase acts on the glycones that contain a compound such as phytoestrogens, polyphenols, isoflavones, biochanin A, formononetin, cumestrol, and lignans as the aglycone. In particular, β-glycosidase can very efficiently act on the glycones comprising an isoflavone as the aglycone. Thus, in one embodiment, the composition is advantageously applied in those contexts wherein the isoflavone glycone is, for example, daidzin, genistin, or glycitin, or an acetyl derivative, succinyl derivative, or malonyl derivative thereof.

β-glycosidase is generally classified as a saccharide-chain hydrolase. However, it exhibits a property different from conventional α- and β-glycosidases. β-glycosidase acts upon a glycoside having a linear or branched saccharide chain composed of one or two or more kinds of saccharides, which are bound through a hydroxyl group in the saccharide chain to a compound other than a saccharide. β-glycosidase recognizes the substrate at the 2′-position and cleaves it, whereby the corresponding disaccharide and an aglycon are formed.

Any combination of saccharides can be recognized as an appropriate substrate for compositions containing β-glycosidase. The combination of saccharides can exhibit a disaccharide structure.

β-glycosidase for use in the invention can be obtained in commercial quantities from Penicillium multicolor. The enzyme may also be obtained and purified from microorganisms that produce β-glycosidase by conventional procedures that are well-known in the art, such as, for example, those described in WO 00/18931.

Protease M:

Some compositions may include Protease M. Protease M is an acid proteolytic enzyme preparation produced by Aspergillus oryzae, that is used to hydrolyze food products such as soy, rice, and casein. Protease M has been further characterized and it has been found that, in addition to protease activity, Protease M exhibits glucosidase activity, β-glycosidase activity, lipase activity, amylase activity, glucoamylase activity, xylanase activity, and pectinase activity. For example, Protease M has been found to exhibit a glucosidase activity of about 40 to about 70 u/g; a β-glycosidase activity of about 0.3 to about 0.9 u/g; a protease activity of about 4,000 to about 8,000 u/g; a lipase activity of about 300 to about 500 u/g; an amylase activity of about 160,000 to about 190,000 u/g; a glucoamylase activity of about 24,000 to about 28,000 u/g; a xylanase activity of about 11,000 to about 14,000 u/g; and a pectinase activity of about 40 to about 120 u/g. Moreover, Protease M exhibits an activity on certain glycones that is distinct from the activity of the β-glycosidase enzyme, as described below.

The relative activity of Protease M and β-glycosidase on different sugar substrates was assessed. A panel of substrates comprising a nitrophenyl group conjugated to different sugars was used in the study. Table 1 below shows the relative activity Protease M had for each substrate (with 100 activity “units” arbitrarily chosen for the substrate on which Protease M exhibited the most activity) and the relative activity β-glycosidase had for each substrate (with 100 activity units arbitrarily chosen for the substrate on which β-glycosidase exhibited the most activity). As seen in Table 1, Protease M exhibits a different activity profile, and different relative substrate activity, than β-glycosidase.

TABLE 1

| Compound | Protease M | β-glycosidase |
|---|---|---|
| 4-Nitrophenyl α-L-arabinopyranoside | 29 | 60 |
| 4-Nitrophenyl β-D-galactopyranoside | 100 | 85 |
| 4-Nitrophenyl α-D-glucopyranoside | 42 | 19 |
| 4-Nitrophenyl β-D-maltoside | 63 | 57 |
| 4-Nitrophenyl β-D-cellobioside | 68 | 55 |
| 4-Nitrophenyl β-D-glucuronide | 4 | 12 |
| 4-Nitrophenyl α-D-galactopyranoside | 6 | 100 |
| 4-Nitrophenyl β-D-mannopyranoside | 10 | 12 |
| 4-Nitrophenyl α-D-mannopyranoside | 4 | 19 |

The invention also includes enzyme compositions exhibiting an enzyme activity profile similar to that of Protease M. For example, enzyme compositions exhibiting one or more of glucosidase activity, β-glycosidase activity, protease activity, lipase activity, amylase activity, glucoamylase activity, xylanase activity, and pectinase activity is also contemplated, including compositions exhibiting one or more such activities at a level comparable to that of Protease M. In one embodiment, the enzyme composition exhibits an activity profile comprising one or more of a glucosidase activity of about 40 to about 70 u/g; a β-glycosidase activity of about 0.3 to about 0.9 u/g; a protease activity of about 4,000 to about 8,000 u/g; a lipase activity of about 300 to about 500 u/g; an amylase activity of about 160,000 to about 190,000 u/g; a glucoamylase activity of about 24,000 to about 28,000 u/g; a xylanase activity of about 11,000 to about 14,000 u/g; and a pectinase activity of about 40 to about 120 u/g. In a specific embodiment, the enzyme composition exhibits an activity profile comprising each of a glucosidase activity of about 40 to about 70 u/g; a β-glycosidase activity of about 0.3 to about 0.9 u/g; a protease activity of about 4,000 to about 8,000 u/g; a lipase activity of about 300 to about 500 u/g; an amylase activity of about 160,000 to about 190,000 u/g; a glucoamylase activity of about 24,000 to about 28,000 u/g; a xylanase activity of about 11,000 to about 14,000 u/g; and a pectinase activity of about 40 to about 120 u/g. One specific, non-limiting example of such a composition has an enzyme activity profile comprising a β-glycosidase activity of about 0.6 u/g; a protease activity of about 6,500 u/g; a lipase activity of about 400 u/g; an amylase activity of about 175,000 u/g; a glucoamylase activity of about 26,000 u/g; a xylanase activity of about 12,500; and a pectinase activity of about 80 u/g.

The enzyme compositions of the present invention may be generated by any of a number of methods. For example, individual enzymes may be combined to achieve the desired enzyme composition with a desired enzyme activity profile. By way of example but not by way of limitation, an enzyme composition may include one or more of a glucosidase enzyme, a β-glycosidase enzyme, a protease enzyme, a lipase enzyme, an amylase enzyme, a glucoamylase enzyme, a xylanase enzyme, and a pectinase enzyme.

Additionally or alternatively, the compositions may be obtained from a microorganism that produces enzymes naturally or that is genetically modified to produce one or more enzymes, using methods well known in the art. For example, Protease M (which exhibits glucosidase activity, β-glycosidase activity, protease activity, lipase activity, amylase activity, glucoamylase activity, xylanase activity, and pectinase activity) can be obtained from Aspergillus oryzae by methods known in the art, and diluted or concentrated prior to use. An exemplary process for Protease M production is outlined below.

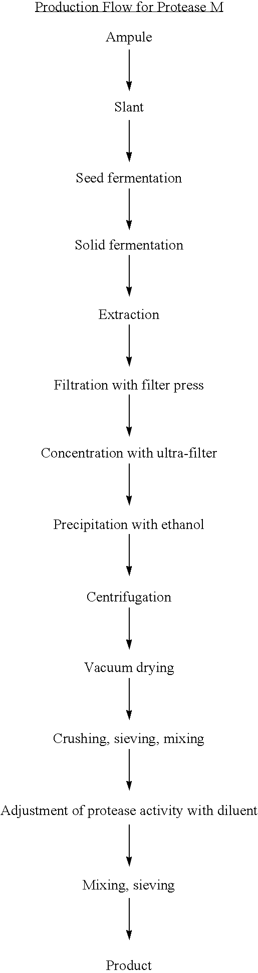

Although this exemplary production flow names Protease M, it will be understood by those skilled in the art that similar production flows may be used to obtain suitable enzyme preparations from other microorganisms.

As noted above, enzymes and enzyme preparations may also be obtained from transformed or transfected cells by methods well known in the art. For example, a nucleic acid sequence encoding a desired enzyme can be inserted into an expression vector, which can be used to transform or transfect a host cell for production of the enzyme. Enzyme can then be obtained from the host cell by methods well known in the art.

Additionally, many enzymes are commercially available. For example, a typical commercial preparation of Protease M (which is commercially available from Amano Enzyme USA, Co., Ltd., Elgin, Ill.) has a protease activity of not less than 5,500 u/g at pH 3.0. This commercial preparation may be used at the given concentration, or the commercial preparation may be diluted or concentrated for use.

The amount of a given enzyme or enzyme activity in a composition according to the invention may vary based on the desired effect of the composition, and may be determined or measured by a variety of method known in the art. The amount of enzymes present in a composition may be stated in molar amounts or molar ratios (e.g., nanomoles or micromoles of enzyme), weight amounts or weight ratio (micrograms or nanograms of enzyme), or activity amounts or activity ratios (e.g., “units” of enzyme or enzyme activity/weight or mole of enzyme). In particular embodiments, compositions may include β-glycosidase, protease, lipase, amylase, glucoamylase, xylanase, and pectinase at defined enzyme activities.

Thus, for example, the composition may comprise an enzyme activity pofile comprising one or more of a glucosidase activity of about 40 to about 70 u/g; a β-glycosidase activity of about 0.3 to about 0.9 u/g; a protease activity of about 4,000 to about 8,000 u/g; a lipase activity of about 300 to about 500 u/g; an amylase activity of about 160,000 to about 190,000 u/g; a glucoamylase activity of about 24,000 to about 28,000 u/g; a xylanase activity of about 11,000 to about 14,000 u/g; and a pectinase activity of about 40 to about 120 u/g. A specific example of a suitable composition has an enzyme activity profile comprising a β-glycosidase activity of about 0.6 u/g; a protease activity of about 6,500 u/g; a lipase activity of about 400 u/g; an amylase activity of about 175,000 u/g; a glucoamylase activity of about 26,000 u/g; a xylanase activity of about 12,500; and a pectinase activity of about 80 u/g. Conventional enzyme assays that are well known in the art can be employed to determine the enzyme activities. These enzyme amounts are exemplary only, and compositions comprising other amounts of enzyme are contemplated.

Other Components

The compositions described herein generally do not and need not contain additives. However, some embodiments provide for the addition of one or more buffers to the compositions. The use of buffers is not necessary, but can help to stabilize pH-sensitive enzymes. Exemplary buffers include but are not limited to acetate buffer and phosphate buffer. Illustrative concentrations of acetate buffer range from about 10 mM to about 100 mM, giving a pH of about 4 to about 6, and of phosphate buffer in the range from about 10 mM to about 100 mM, giving a pH from about 6 to about 8.

II. Food and Beverage Products

Other embodiments of the invention include food and beverage products that comprise or have been treated with an enzyme composition of the present invention.

Many food and beverages contain glycones that yield physiological beneficial aglycones. Although the particular embodiments and examples that follow name specific foods or beverages to demonstrate the utility and diversity of the present invention, it should be understood that the invention is not limited to those foods or beverages, rather, the invention is intended to include all food and beverages.

In some embodiments, the food product is a vegetable or vegetable product. Illustrative vegetables in this regard include garlic, asparagus, peppers, and mushrooms. In one particular embodiment, the vegetable is garlic.

In some embodiments, the food product is a fruit or fruit product. In one embodiment, the fruit is a tomato or a tomato product. Examples of tomato products include but are not limited to tomato purees; tomato pastes; tomato-based sauces; tomato-based juices; and condiments such as, for example, ketchup, salsa and picante sauce; and tomato-containing soups.

Other embodiments are directed to a beverage product. The term “beverage product,” as used herein, refers to any liquid composition fit for human oral consumption, as well as to concentrated forms of such liquid compositions. Suitable beverages include but are not limited to products consisting of or comprising coffee, tea, fruit and vegetable juices, alcoholic beverages, and mixtures thereof. In one embodiment, the beverage is a tea. The tea can be fresh-brewed, for example from tea leaves, or can be prepared from a powder or syrup form (“instant tea”). Thus, the tea also includes concentrated forms of tea such as, for example, powdered tea mixes.

In another embodiment, the beverage is a fruit juice. Specific examples of fruit juices include but are not limited to apple, pomegranate, grape, orange, grapefruit, cherry, blueberry and cranberry juices, and mixtures of these juices. The fruit juice may be fresh, processed (e.g., pasteurized) or from a powder or syrup. When treated with enzymes, some fruit juices form a colored precipitate. While the precipitate does not necessarily affect the flavor of a fruit juice, it can detract from the visual appeal and mouth feel of the fruit juice. The enzyme compositions according to the invention can be used to enhance the flavor of a fruit juice, while avoiding or at least minimizing the formation of such precipitates, thereby increasing the appeal of the fruit juice to a consumer.

Still other embodiments provide for the beverage to be an alcoholic beverage. The alcoholic beverage is any kind of such beverage, for example a wine or beer. In one embodiment, the alcoholic beverage is a wine.

In some embodiments, the enzyme composition is present in the food or beverage product in an amount sufficient to enhance the product flavor. The exact amount of the composition to be added will vary depending on the food or beverage product and the concentration or activity of the enzyme composition used. It should be understood that the flavor of a product includes but is not limited to the taste and aroma characteristics of the product. Enhanced flavor can be assessed by conventional means, such as by the use of professional or non-professional taste testers.

In other embodiments, the enzyme composition is present in the food or beverage product in an amount sufficient to increase the aglycone content of the food or beverage product, relative to the same food or beverage that has not been treated with or contacted with the enzyme composition. The level of aglycone present in a food or beverage before and after enzyme treatment may be determined empirically and can be measured by any conventional means, such as by routine chemical analysis (e.g., HPLC, etc.).

In general, the enzyme compositions of the invention (such as compositions comprising one or more of glutaminase, β-glycosidase, protease, lipase, amylase, glucoamylase, xylanase, pectinase, RPI, deaminase, and glucosidase or exhibiting one or more of those enzyme activities) may be present in a concentration of up to about 3% (w/v), up to about 2% (w/v), or up to about 1% (w/v). Specific concentrations that may be used are about 0.01% (w/v), about 0.02% w/v, about 0.025% (w/v), about 0.04% w/v, about 0.05% (w/v), about 0.06% w/v, about 0.08% w/v, or about 0.10% w/v, such as 0.01% w/v, 0.02% w/v, 0.025% (w/v), 0.04% w/v, 0.05% (w/v), 0.06% w/v, 0.08% w/v, or 0.10% w/v. These amounts are exemplary only, and food and beverage products comprising different amounts of the composition are also contemplated.

III. Process of Making Flavor- and/or Nutritionally-enhanced Food or Beverage Products

Other embodiments are directed to a process for producing a food or beverage product. In these embodiments, the process comprises contacting the food or beverage product with a flavor-enhancing amount of an enzyme composition of the invention, or with an amount of the enzyme composition effective to increase the aglycone content of the food or beverage product, relative to the same food or beverage that has not been treated with the enzyme composition. Another embodiment is a food or beverage product that is made by this process.

In some embodiments, the enzyme composition is simply contacted in undiluted form with the food or beverage, such as by mixing or blending the composition into the product, or by spraying the composition onto the product. In this regard, the process as described herein imposes few additional requirements on the manufacture of food and beverage products. In some embodiments, as mentioned above, one or more buffers can be added with the composition, although this is not usually necessary.

In other embodiments, the enzyme composition is added to one or more raw ingredients of the food or beverage product, such as during the manufacturing process of the food or beverage product.

In some embodiments, the process provides, as an additional and sequential step, for the enzymes in the composition described herein to be inactivated by heating the resultant food or beverage product for a time that is sufficient to inactivate one or more of the enzymes (or enzyme activities) present in the composition. The temperatures and times required to achieve this post-processing inactivation will vary, and can be empirically determined for a given food or beverage product. Exemplary temperatures can range from about 70° C. to about 90° C. Exemplary times can range from about 5 to about 60 minutes and from about 5 to about 30 minutes. In any case, the time and temperature can be chosen such that enzyme activity is reduced or eliminated to the desired extent and such that the inactivation step does not degrade or otherwise compromise the desired food or beverage product. These embodiments may be advantageous because inactivation of one or more enzymes prevents extended enzymatic action that may occur, such as upon storage and/or transport of the product, that may lead to the buildup of undesirable flavors that might develop as a result of extended enzyme activity.

### EXAMPLES

The following examples are given to illustrate the present invention. It should be understood, however, that the invention is not to be limited to the specific conditions or details described in these examples. All publicly available documents, including U.S. patents, cited herein are incorporated by reference in their entireties as if fully set forth herein.

### Example 1

### HPLC Analysis of Cranberry Juice

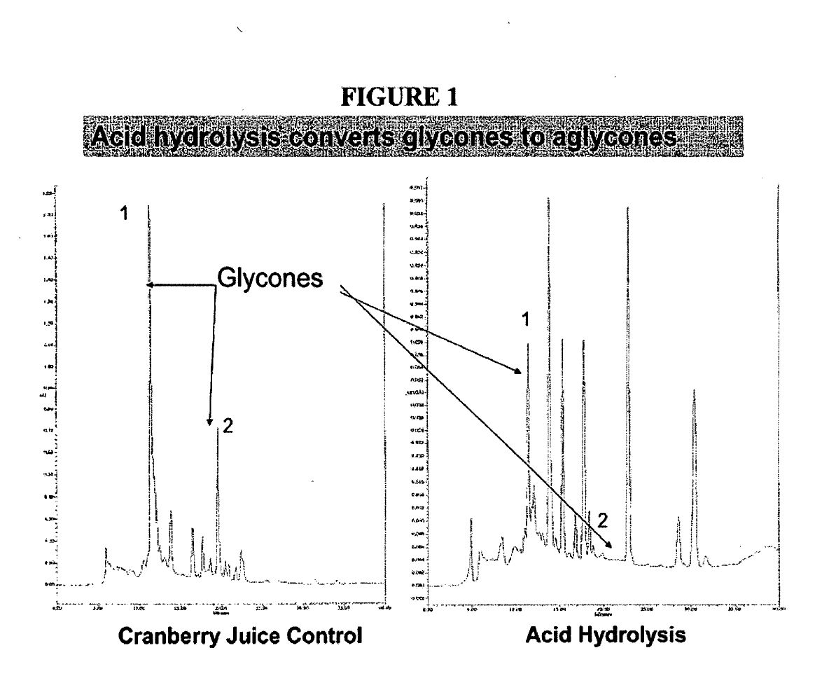

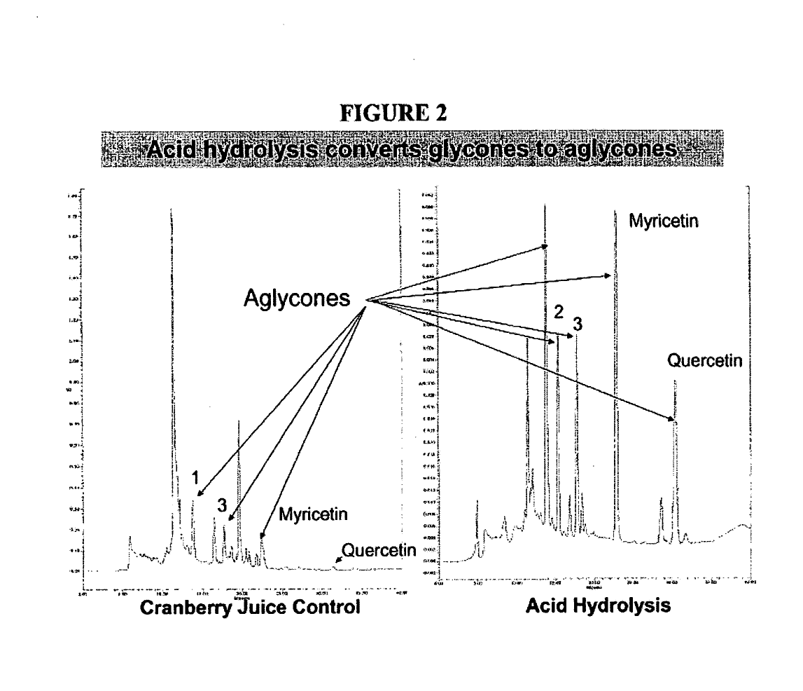

HPLC (high-performance liquid chromatography) analysis of untreated cranberry juice reveals a pattern of peaks, each attributable to a specific glycone (flavanoid) or aglycone present in the juice. (FIGS. 1 & 2, Left Panels). Acid hydrolysis of cranberry juice reduces the glycone content while increasing the aglycone content (also shown by characteristic HPLC peaks ). (FIGS. 1 & 2, Right Panels). Acid hydrolysis was performed as follows: HCl was added to a final concentration of 0.6M. The sample was vortexed and then incubated at 90° C.-95° C. for one hour. The sample was then centrifuged at 7500 RPM for 5 minutes, and analyzed by HPLC.

### Example 2

### Treatment of Cranberry Juice with Protease M or β-glycosidase and HPCL Analysis

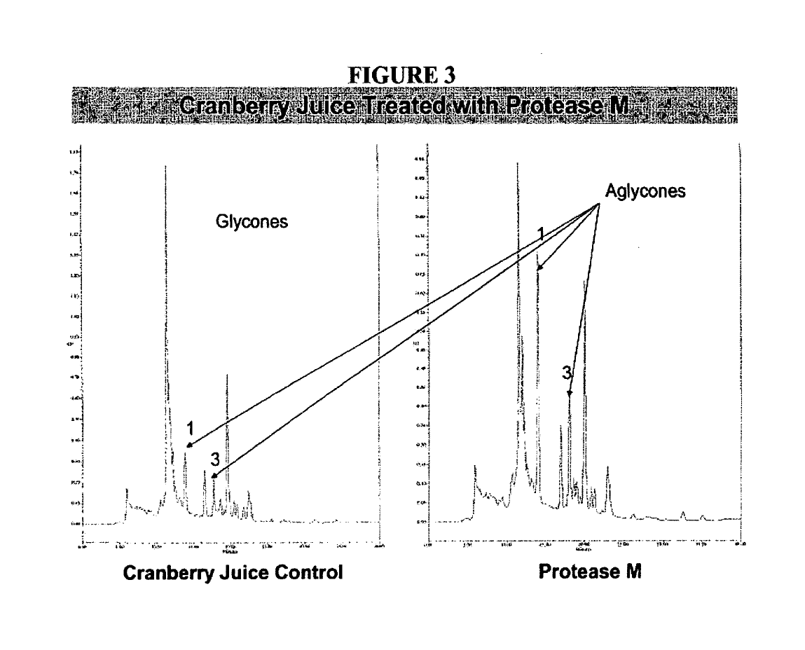

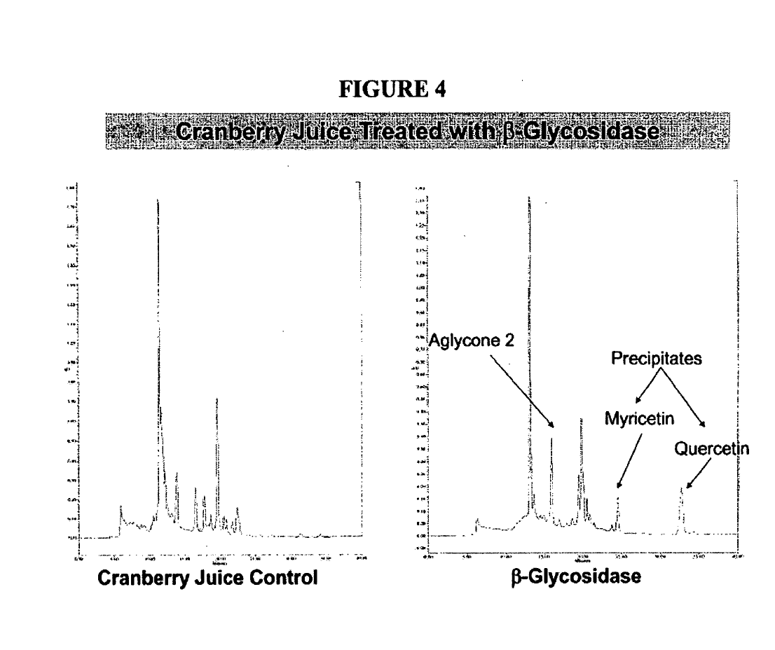

As shown in FIG. 3, Protease M (3 hour treatment at 50° C. with 0.1% (w/v) Protease M) acts on glycones 1 and 3 of cranberry juice, yielding a product with corresponding aglycones 1 and 3. In contrast, as shown in FIG. 4, treatment with β-glycosidase (3 hour treatment at 50° C. with 0.1% w/v β-glycosidase) acts on glycone 2 of cranberry juice and the glycones corresponding to myricetin and quercetin, yielding a product with corresponding aglycone 2 and precipitates of myricetin and quercetin.

The effects of Protease M on the glycone/aglcyone profile of cranberry juice could be particularly advantageous. For example, the increase in aglcyone levels in the treated juice represents an increase in the bioavailability of the flavanoids, and directly correlates with an increase in the antioxidant potential of the treated juice. Similar benefits can be obtained by treating other foods and beverages with Protease M.

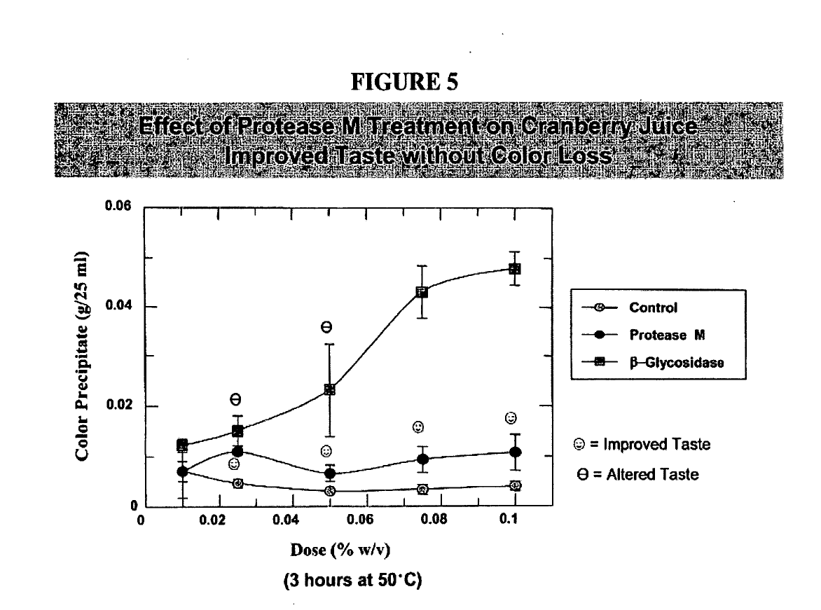

As shown in FIG. 5, cranberry juice treated with Protease M maintains its color and exhibits enhanced flavor relative to untreated cranberry juice (control). In contrast, cranberry juice treated with β-glycosidase yields colored precipitates (resulting in color loss from the juice) and was found to have an altered taste relative to untreated cranberry juice. Cranberry juice was treated with Protease M or β-glycosidase at does of 0.02, 0.04, 0.06, 0.08 and 0.1% w/v and results were observed after 3 hours at 50° C. Similar benefits can be obtained by treating other foods and beverages with Protease M.

### Example 3

### Treatment of Cranberry Juice with Protease M and Taste Test

A variety of commercial cranberry juice products were treated with 0.1% w/v of Protease M at 50° C. for 3 hours, cooled in a refrigerator and used in a taste test. Five tasters were used, and the reported results reflect a consensus. As shown in Table 1 below, the Protease M-treated products were found to have enhanced flavor over untreated juice. In particular, the Protease M-treated products consistently were found to have a sweeter, less tart flavor. These same results were obtained with 150 different tasters. This enhanced flavor property of Protease M-treated cranberry juice could be particularly advantageous. For example, cranberry juice products treated with Protease M could be formulated with less sugar (or other sweeteners, including other sweeter juices) and still be palatable or have a more acceptable level of tartness. Such products would have clear benefits for subjects limiting their sugar intake or limiting their caloric intake. Similar benefits can be obtained by treating other foods and beverages with Protease M.

TABLE 1

| | Dose | |
|---|---|---|
| Product | (% w/v) | Taste Test Results |
| Ocean Spray Premium | 0.1 | Sweeter, fruiter flavor, treated |
| 100% Cranberry Juice | | juice preferred |
| (cranberry and mixed berry) | | |
| Ocean Spray | 0.1 | Slight reduction in color, not as |
| Cran-Apple | | tart, sweeter, mostly tastes of |
| | | apple |
| Ocean Spray | 0.1 | Reduces sourness, slightly |
| Cranberry Juice and Tea | | sweeter |
| Ocean Spray | 0.1 | Enhanced cranberry flavor, |
| Cran-Tangerine | | reduced tartness |
| Libby's Juicy Juice | 0.1 | Sweeter, very mild |
| Cranberry Apple | | |
| Old Orchard | 0.1 | Better blend of flavors, sweeter, |
| Cranberry Raspberry | | more mellow |
| Juice Cocktail Blend | | |

Accordingly, results indicate that Protease M treatment removes the bitterness in cranberry juice. Further, the formation of aglycones does not lead to color loss or precipitation. Additionally, the increase in aglycone levels after Protease M treatment increases the antioxidant potential of the juice and increases the bioavailability of protective flavonoids.

### Example 4

### HPLC Analysis of Treated Grape, Cherry and Blueberry Juice

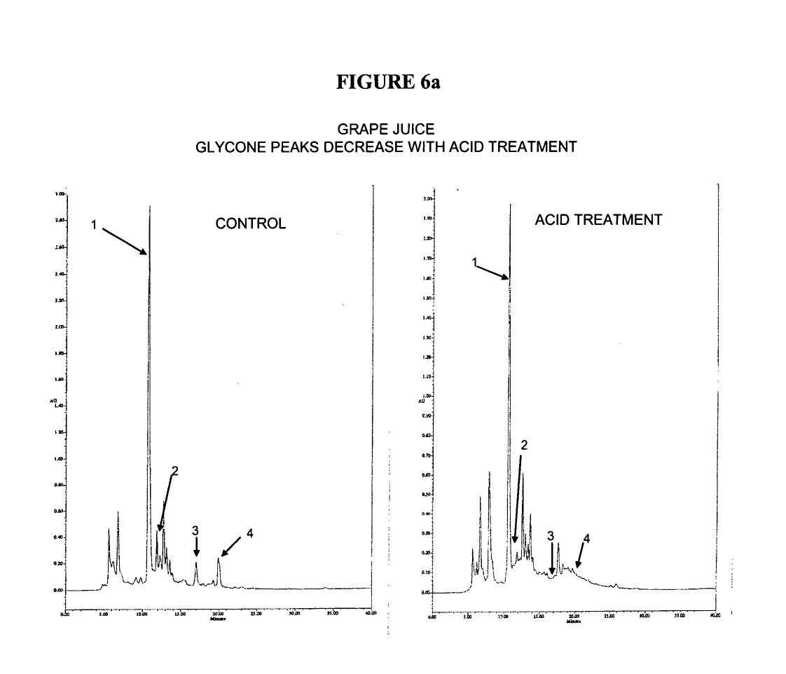

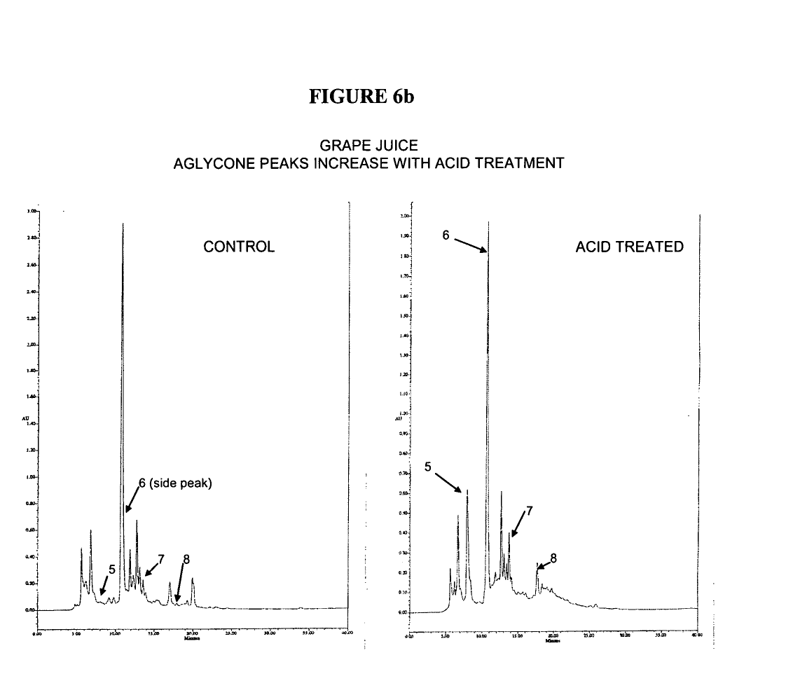

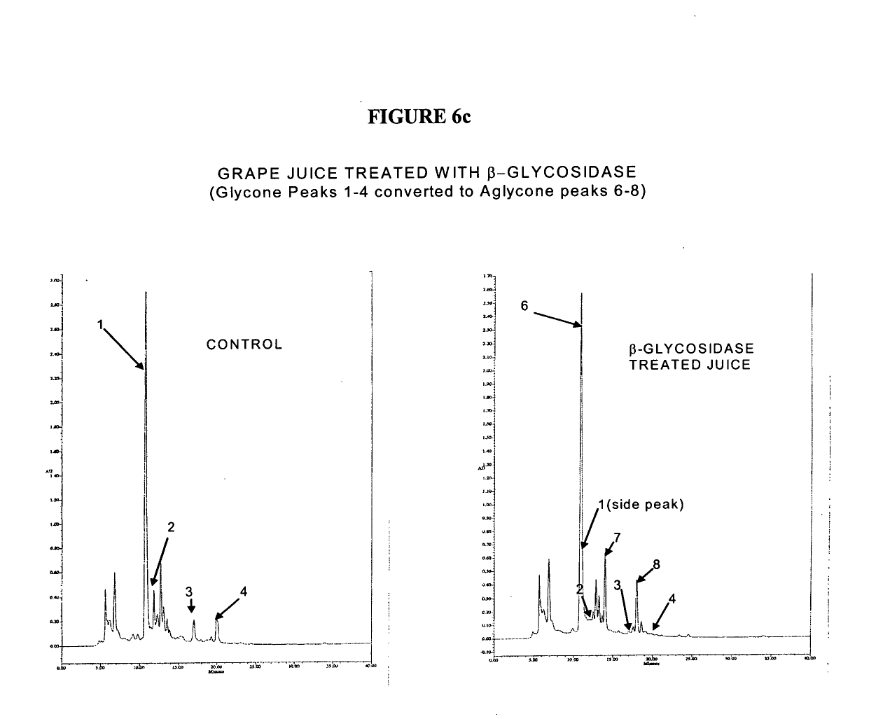

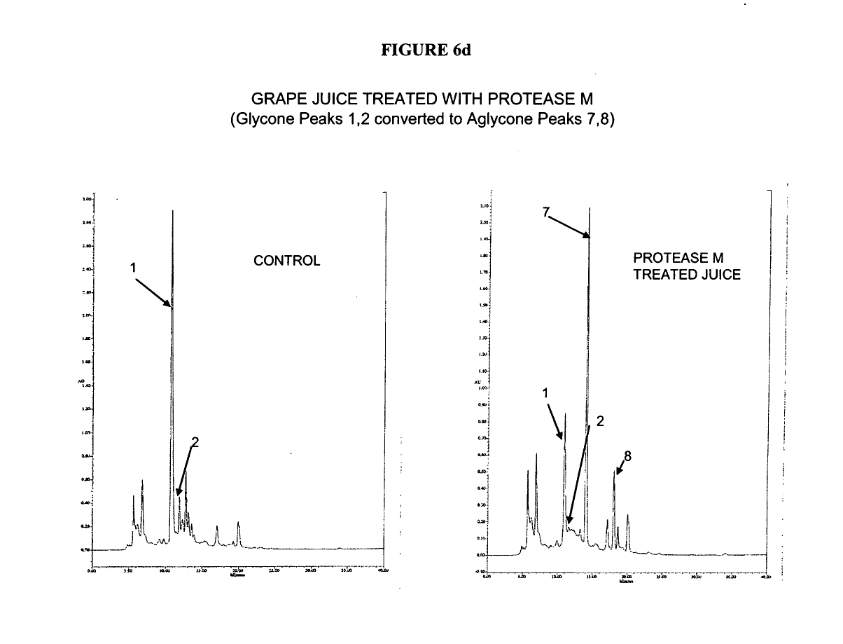

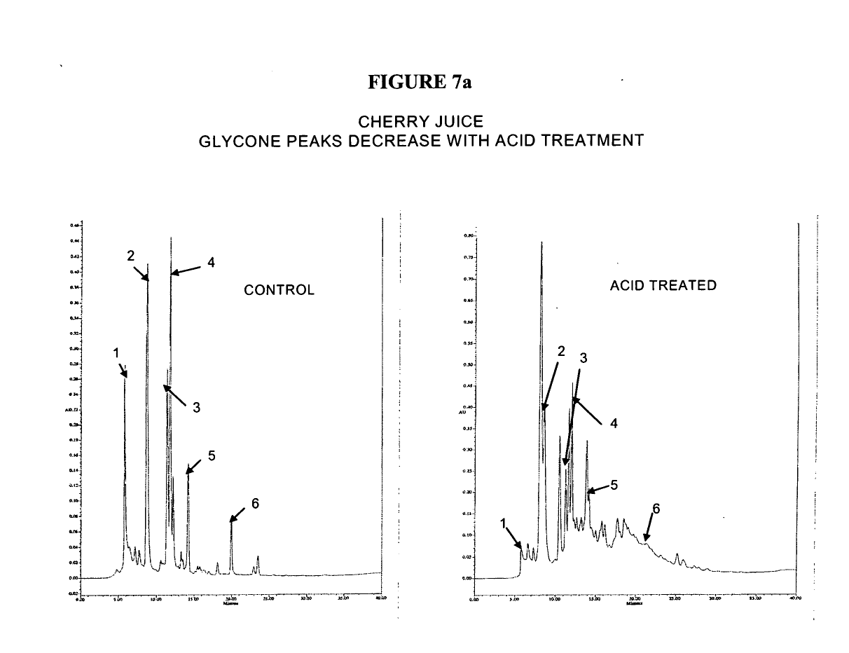

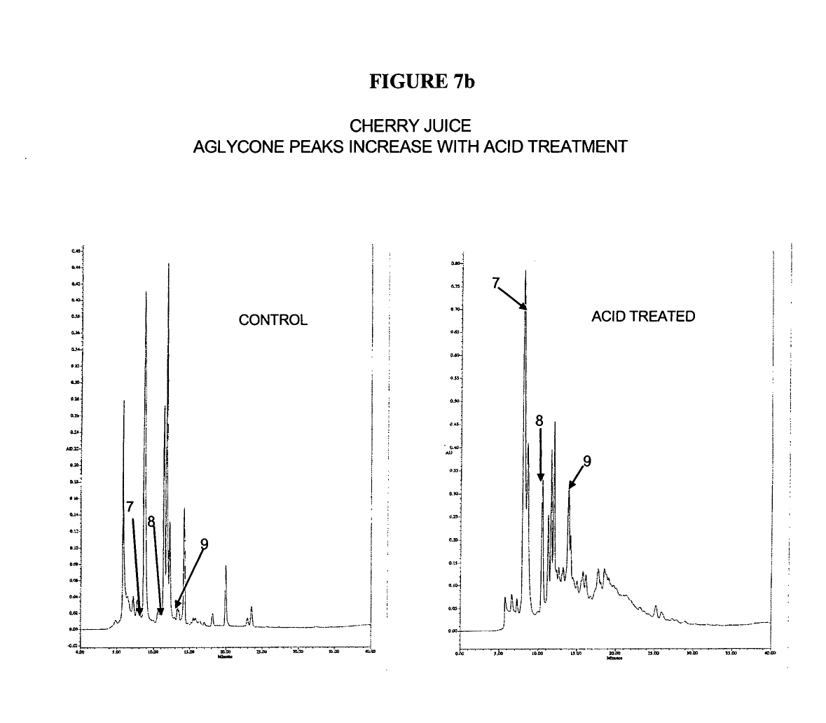

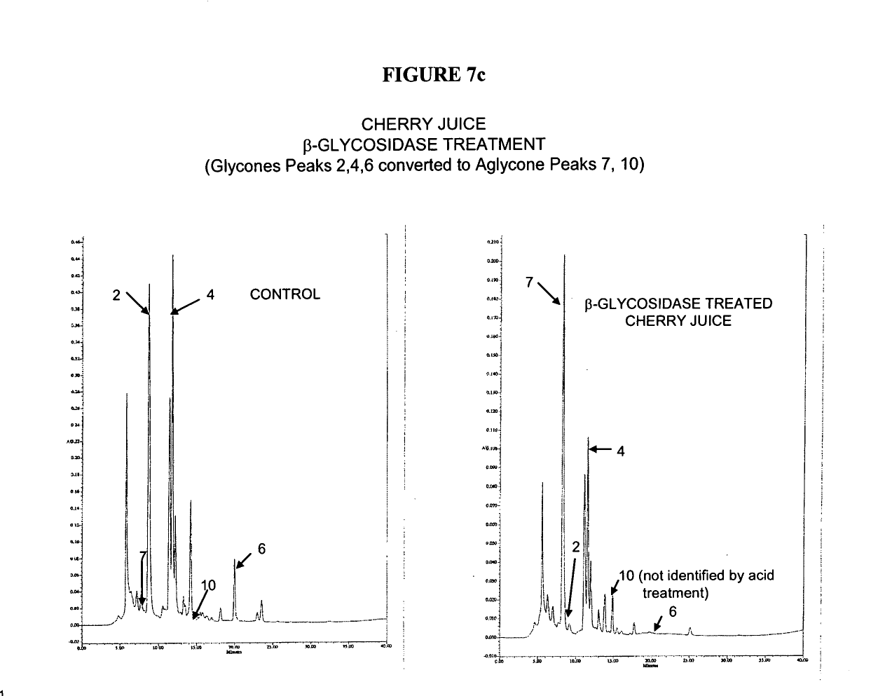

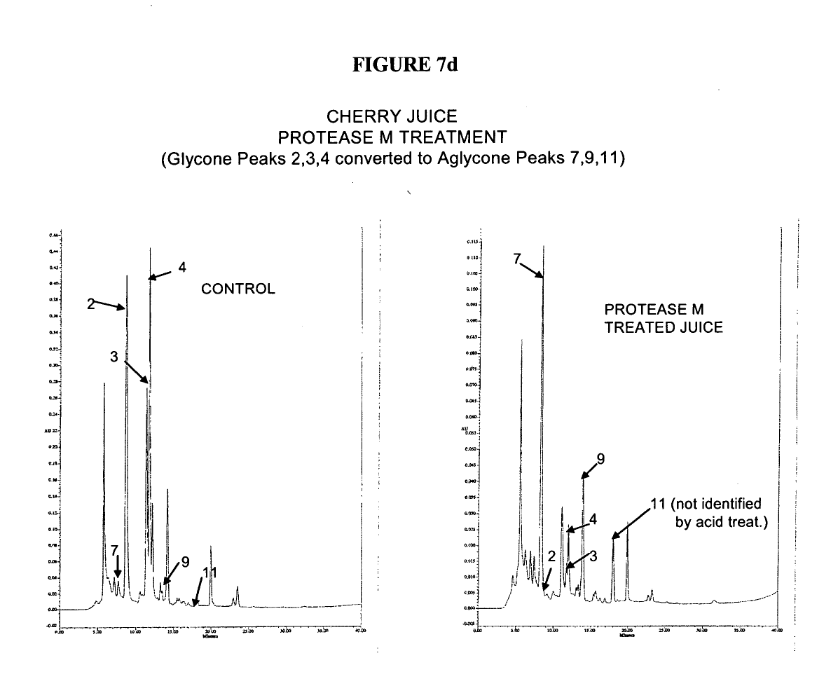

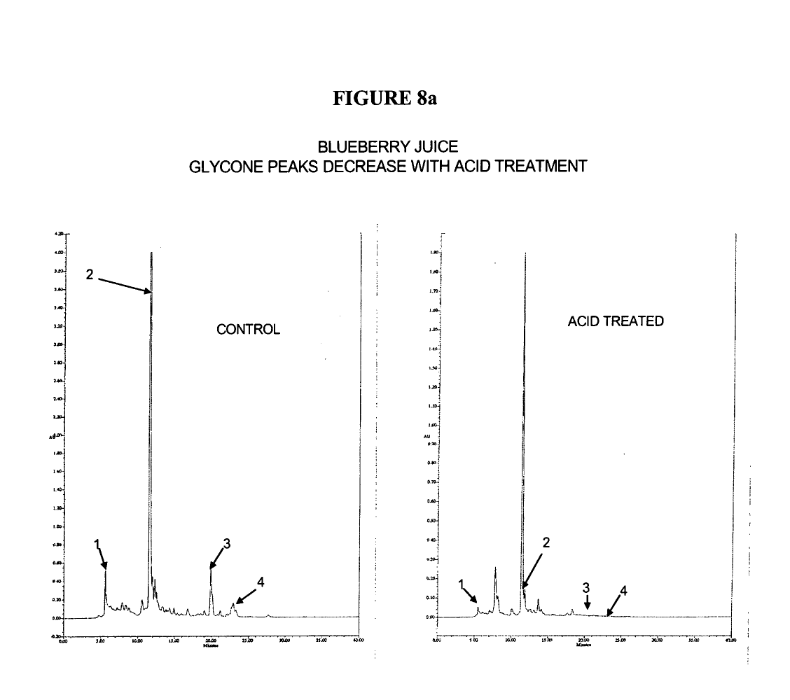

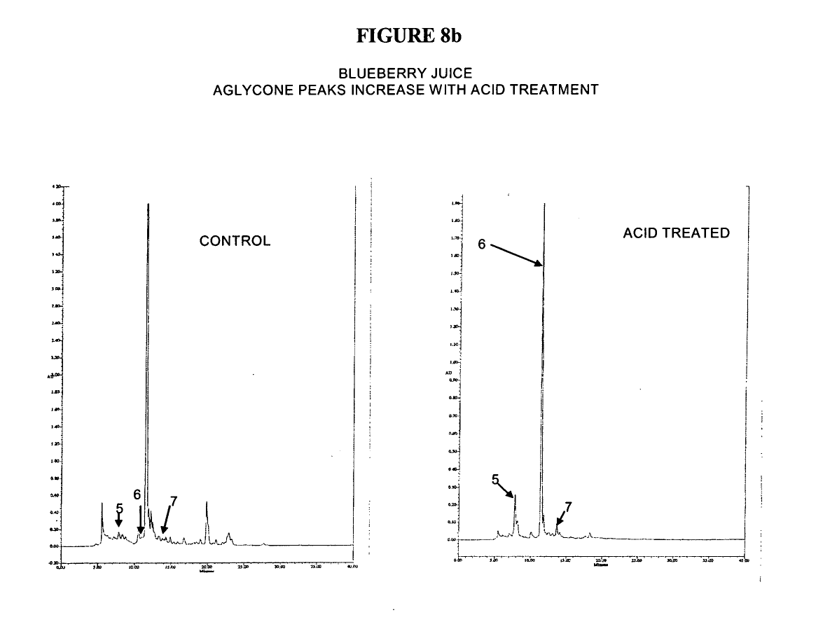

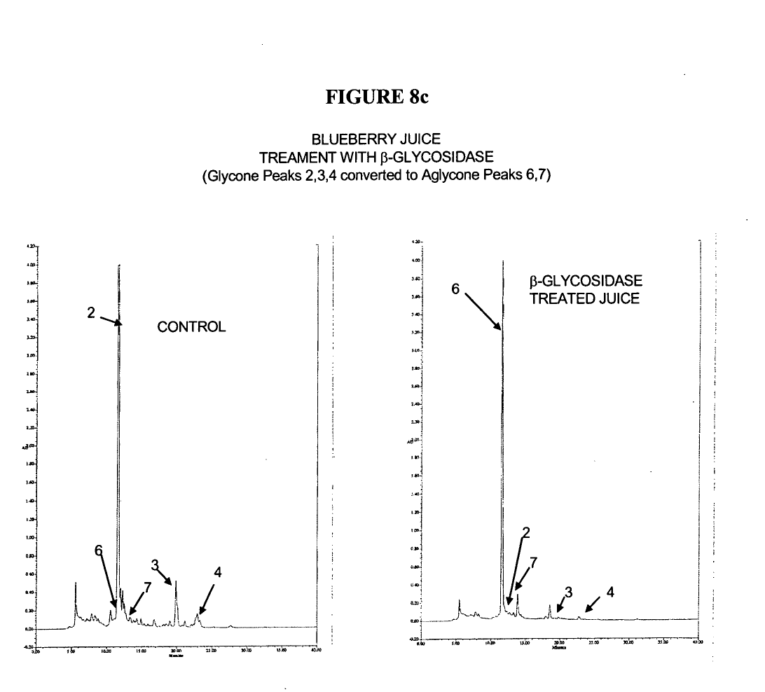

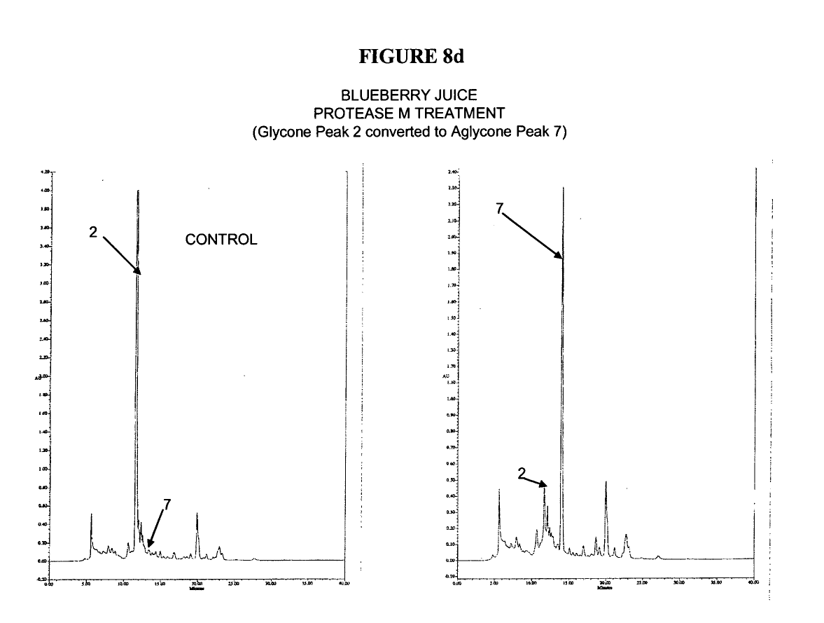

Aliquots of grape, cherry and blueberry juice were subject to acid hydrolysis as described in Example 1, treated with β-glycosidase as described in Example 2, or treated with Protease M as described in Example 2. Chromatographs comparing the untreated juice (control) with the treated samples are shown in FIGS. 6-8. In all cases, glycone peaks are diminished in the treated as compared to untreated samples, and glycone peaks are converted to aglycone peaks in the treated juice samples.

### Example 5

### Treatment of a Variety of Juices with Protease M and Taste Test

A variety of commercial juice products (including vegetable, grapefruit, orange and apple juices) were treated with 0.1% w/v of Protease M for 3 hours at 50° C. and used in a taste test. As shown in Table 2 below, the Protease M-treated products were found to have enhanced flavor over untreated juice. In particular, Protease M was found to enhance the flavor of the tomato juice products, reduce the bitterness of the grapefruit juice product, enhance the flavor of the orange juice product, and increase the sweetness and apple flavor of the apple juice product. Similar benefits can be obtained by treating other foods and beverages with Protease M.

TABLE 2

| | Dose | |
|---|---|---|
| Product | (% w/v) | Taste Testing Results |
| Campbell's V8 | 0.1% | More intense vegetable taste, |
| 100% Vegetable Juice | | saltier, more mouth feel and |
| | | richer taste. |
| Campbell's V8 | 0.1% | More mouth feel, more balanced |
| Low Sodium Vegetable | | taste |
| Juice | | |
| Campbell's V8 | 0.1% | Increased spiciness overpowered |
| Spicy Hot 100% Vegetable | | milder tomato flavor. More |
| Juice | | intense flavor overall. |
| Tropicana Premium | 0.1% | Low dose (0.01%) reduced |
| Ruby Red Grapefruit Juice | | sourness (but bitter aftertaste still |
| | | present). Higher dose removed |
| | | the bad aftertaste and was |
| | | preferred. |
| Florida's Natural | 0.1% | Higher dose increased mouth feel |
| No Pulp Original Orange | | and flavor. |
| Juice | | |
| Old Orchard | 0.1% | Sweeter, increased apple flavor. |
| 100% Apple Juice | | |

### Example 6

### Treatment of a Variety of Juices with Protease M or β-Glycosidase and Taste Test

A variety of juices were treated with either water (control), 0.1% w/w Protease M, or 0.1% w/w β-glycosidase as described above in Example 2. The juices were chilled in a refrigerator before tasting.

Results are shown below in Table 3. In most cases, tasters noted that the flavor of the juice is different after treatment with the enzyme preparations, and that in some cases, the flavor after Protease M treatment is preferred, while in other cases, the flavor after β-glycosidase treatment may be preferred. For example, in some instances, the β-glycosidase produced a “floral” aroma, which may be preferred for tea, wine or other foods and beverages.

TABLE 3

| Juice | Treatment | Color Loss | Aroma/Taste Results |
|---|---|---|---|
| Bionaturae Organic | Control | 0.9014 | Like cherry candy flavor, |
| Sour Cherry Nectar | (water) | | cloying, little aroma, some tartness |
| Bionaturae Organic | Protease M | 0.6041 | Richer, deeper, more |
| Sour Cherry Nectar | 0.1% w/w | | complex cherry flavor, |
| | | | stronger aroma than |
| | | | control, decreased |
| | | | tartness |
| Bionaturae Organic | β-glycosidase | 0.9873 | Increased flavor |
| Sour Cherry Nectar | 0.1% w/w | | intensity, less sweet |
| | | | (more tart than Protease |
| | | | M), floral note |
| R. W. Knudsen | Control | 0.0594 | Some aroma (not very |
| Just Blueberry | (water) | | strong), thin (little |
| Juice | | | mouthfeel), tart, sharp, |
| | | | not sweet, little to |
| | | | moderate flavor |
| R. W. Knudsen | Protease M | 0.0559 | Aroma more like |
| Just Blueberry | 0.1% w/w | | blueberries, increased |
| Juice | | | mouthfeel, decreased |
| | | | tartness, increased |
| | | | sweetness and flavor, |
| | | | “fresher” taste-more |
| | | | like fresh blueberries |
| R. W. Knudsen | β-glycosidase | 0.0670 | floral note and aroma, |
| Just Blueberry | 0.1% w/w | | darker color (less purple), |
| Juice | | | decreased tartness, sweet, |
| | | | doesn't taste as “blueberry-like” |
| | | | as Protease M juice |
| R. W. Knudsen | Control | 0.0809 | Little aroma, bitter, very |
| Just Cranberry | (water) | | sour, hard to tell flavor |
| Juice | | | |
| R. W. Knudsen | Protease M | 0.0536 | Increased cranberry |
| Just Cranberry | 0.1% w/w | | aroma, less bitter, still |
| Juice | | | sour, increased flavor |
| | | | than control |
| R. W. Knudsen | β-glycosidase | 0.0856 | Raspberry-like aroma, |
| Just Cranberry | 0.1% w/w | | bitter, much less sour, |
| Juice | | | some sweetness, better |
| | | | flavor than control but |
| | | | less than Protease M |
| | | | juice |
| Welch's 100% | Control | 0.0610 | Little aroma, slightly |
| Grape Juice | (water) | | sweet |
| Welch's 100% | Protease M | 0.0827 | Grape aroma, sweeter, |
| Grape Juice | 0.1% w/w | | more aftertaste (taste |
| | | | lingers longer in mouth), |
| | | | flavor like grape jam |
| Welch's 100% | β-glycosidase | 0.1139 | Sweeter aroma, very |
| Grape Juice | 0.1% w/w | | strong floral note, much |
| | | | sweeter than Protease M |
| | | | juice, like grape soda |

### Example 7

### Enzymatic Treatment of Tomato Paste

Preparation: Tomato paste (200.40 g, 40% dissolved solid) was mixed thoroughly with 600 ml water to bring dissolved solid (ds) to about 10%. The initial pH of the resulting mixture was 4.36, which was adjusted pH 6.01 with about 50 ml 1 M NaOH. Three 200 ml aliquots (A, B, and C) were poured into separate sterile flasks. Samples B and C were stored in a cold cabinet.

Enzyme solutions were prepared by dissolving 1.00 g each of (1) a mixture of β-glycosidase, protease, lipase, amylase, glucoamylase, xylanase, and pectinase; (2) glutaminase F100; and (3) β-glycosidase in 10 ml water volumes.

Flask A was dosed with 2.0 ml of solution (1) and 2.0 ml of solution (2) (0.1% w/w doses each). The sample was then incubated at 50° C. and 300 RPM for 3 h. At end of incubation, the pH of the mixture was adjusted to 4.45 with 1 M HCl, and the mixture was placed in a 70° C. bath for 1 hr to inactivate enzyme.

Sample B was dosed with 2.0 ml glutaminase (solution (2)) and 2.0 mL β-glycosidase (solution (3)) (0.1% w/w doses each). Sample C was treated with 4.0 ml of water as a control. Both samples were incubated at 60° C. and 300 RPM for 3 hr. At end of incubation, the samples were treated similarly as above to adjust for pH and inactivate enzyme described.

Tasting: All samples were warmed in a 50° C. bath for at least 15 minutes prior to the taste test and the samples were tasted without dilution. The samples were given to four tasters; all of them thought Sample A had more tomato flavor while sample B had more mouthfeel. The samples were frozen for about two weeks, thawed, and warmed at 70° C. for 15 minutes. A second group of tasters preferred Sample A as having enhanced tomato flavor.

### Example 8

### Enzymatic Treatment of Garlic

Preparation: Several bulbs of commercially available garlic (Frieda's Elephant Garlic) were peeled, chopped, into pieces with a knife, and then processed in a food processor until a creamy paste was formed. Three 50.0 g portions of the garlic paste were weighed into sterile flasks labeled A, B, and C. Samples B and C were temporarily stored in a cold cabinet. 1.00 g samples each of glutaminase F100; an enzyme composition comprising β-glycosidase, protease, lipase, amylase, glucoamylase, xylanase, and pectinase; and β-glycosidase, were weighed and dissolved as described above in Example 1.

Sample A was dosed with 0.25 ml of the glutaminase solution and 0.25 ml of the enzyme composition solution, where doses for both enzymes were 0.05% w/w. The mixture was incubated at 50° C. and 300 RPM for 3 hr. At the end of incubation, the mixture was placed in a placed in a 70° C. bath for 1 hr to inactivate enzyme. Sample B was treated with 0.25 ml β-glycosidase solution and 0.25 ml glutaminase solution (0.05% w/w doses each). 0.5 ml water was added to sample C as a control. Samples B and C were incubated at 60° C. and 300 RPM for 3 hr, then placed in a 70° C. bath for 1 hr to inactivate enzyme.

Tasting: A jar of pasta sauce (Prego® Traditional) was warmed along with separate garlic samples that were treated according to Example 1 (samples A, B, and C). Both pasta sauce and garlic samples were warmed for at least 15 minutes. Treated garlic samples (2.00 g) were brought to volume with 50 mL pasta sauce. The resulting garlic and sauce samples were mixed and given to four tasters. All tasters agreed that samples A and B had a stronger garlic taste than C. Sample A was considered strongest by at least one taster; Sample B was considered sharper.

### Example 9

### Enzymatic Treatment of Mushrooms

Mushrooms (Monterey Clean N Ready Sliced Mushrooms®) were finely chopped using mechanical means (Cusinart Mini-Prep Blender®). Two 100-g portions of the chopped mushrooms were weighed separately into sterile flasks labeled A and B. Sample A was dosed with 1 mL water as control. Sample B was dosed with 1 mL of a solution of enzyme RP-1 (concentration was 0.1 g/ml for a 0.1% w/v dose). The mushroom and enzyme mixture was shaken to mix, then incubated in a 70° C. bath for 3 hr without additional shaking, and then transferred to 80° C. bath for 2 hr to inactivate enzyme. The treated mushrooms were stored in cold cabinet prior to taste test; samples were not warmed up before being tasted. Three of four tasters preferred the treated sample to the control, as having enhanced flavor.

### Example 10

### Enzymatic Treatment of Food and Beverage Products with Enzyme Mixture and Taste Tests

A variety of food and beverage products listed in Table 4 below were treated with Protease M. Table 4 also presents the resulting taste and physical characteristics of the treated products.

A. Solid and Semi-solid Products

Solid and semi-solid products were processed in a manner analogous to the procedures described in Examples 7 and 8 above. The product samples were prepared using concentrations of the Protease M composition at 0.01, 0.025, 0.05, and 0.1% w/v. Control samples contained no enzyme composition.

B. Beverage Products

Beverage products were treated with the Protease M enzyme composition according to the following procedure.

500-g aliquots of the beverage product were weighed into separate sterile flasks. Aliquots of the Protease M composition as an aqueous solution (100 mg/mL) were added to each flask, where the concentrations of the composition used were 0.01, 0.025, 0.05, 0.075, and 0.1% w/v. Each flask was shaken to mix, then incubated at 50° C. and 165 RPM for 3 hr. The enzymes were then inactivated by heating the beverage products to 70° C. for 1 hr. Each sample was centrifuged in a tube, and the collected precipitate was weighed after drying.

The remaining beverage products were cooled in a refrigerator until they were ready for the taste tests.

C. Taste Tests

The tastes of food and beverage products presented in Table 4 below were evaluated by three to five people, each of whom sampled no more than three samples during any one taste test. One sample was the control or untreated sample and the other two samples were treated with enzyme (Protease M). Water was provided to the evaluators to remove taste in between samples. Most samples were tasted as is, except that garlic was added to tomato paste (2 g per 50 mL) for tasting. Juice was tasted after it was chilled in a refrigerator and tomato paste was tasted warm immediately after heat inactivation of enzyme.

The taste was considered enhanced if all, or no more than one dissenter, clearly detected an enhancement in taste. The characterization of the enhancement was a consensus of the descriptions of the taste testers.

TABLE 4

| | | |
|---|---|---|
| Enzyme Concentration | | |
| (% w/v) | Taste | Physical Characteristics |

Asparagus

| | | |
|---|---|---|
| None | Mild flavor, bitter aftertaste | None noted |
| 0.01 | Increased flavor and bitterness | None noted |
| 0.025 | Intermediate flavor and bitterness, | None noted |
| | gritty | |
| 0.05 | Good flavor, reduced bitterness | Noted softness |
| 0.1 | Similar to 0.05% but with more | Similar to 0.05% |
| | bitterness | |

Green Peppers

| | | |
|---|---|---|
| None | Slightly sweet, mild, not too hot | Crunchy |
| 0.01 | Same as control | Same as control |
| 0.025 | Similar to control with “green note” | Same as control |
| 0.05 | Sweeter, “grassy” - like an unripe | Same as control |
| | green tomato | |
| 0.1 | Sweet, “green note” | soft |

Mushrooms

| | | |
|---|---|---|
| None | Slightly bitter, woody | |
| 0.01 | Odd note (musky) | Darker than control |
| 0.025 | Odd note still there but reduced | Darker than control |
| 0.05 | Odd note | Darker than control |
| 0.1 | Odd note, milder, tastes like cheese | Darker than control |

Red Peppers

| | | |
|---|---|---|
| None | Slightly sweet, mild | Crisp |
| 0.01 | Slightly sour | Watery |
| 0.025 | Stronger than control | Watery |
| 0.05 | Increased flavor | Watery, redder than |
| | | control |
| 0.1 | Stronger flavor, more sour than | Watery, redder than |
| | control | control |

Garlic

| | | |
|---|---|---|
| None | Moderate garlic flavor | None noted |
| 0.01 | Moderate garlic flavor | None noted |
| 0.025 | Garlic itself strong, but sauce doesn't | None noted |
| | have strong garlic flavor | |
| 0.05 | Similar to 0.025 | None noted |
| 0.1 | Similar to 0.025 | None noted |

Pomegranate/Cranberry Juice

| | | |
|---|---|---|
| None | Somewhat sweet, slightly tart, no | None noted |
| | particular flavor | |
| 0.01 | Sweeter, less tart | None noted |
| 0.025 | Sweeter than 0.01%, very mellow | None noted |
| 0.05 | Sweeter than 0.025%, increased | None noted |
| | flavor | |
| 0.1 | Sweeter, more flavor (like apple) | Darker than control |

Cran-Raspberry Juice

| | | |
|---|---|---|
| None | Tart, more raspberry than cranberry | None noted |
| 0.01 | Increased raspberry, decreased | None noted |
| | tartness | |
| 0.025 | Mellow, good raspberry flavor | None noted |
| 0.05 | Mellow, more “blended” flavor | None noted |
| 0.1 | Mellow, “blended” flavor, almost too | None noted |
| | sweet | |

Chunky Tomato, Garlic, and Onion Pasta Sauce (Ragu ®)

| | | |
|---|---|---|
| None | Mild flavor | None noted |
| 0.01 | Spicier | None noted |
| 0.025 | Chunks of vegetables, but not the | None noted |
| | sauce, had more flavor | |
| 0.05 | Spicier than 0.025% | None noted |
| 0.1 | Most flavor, strongest spice, | None noted |
| | considered the best | |

Mushroom and Garlic Pasta Sauce (100% Natural Prego ®)

| | | |
|---|---|---|
| None | Moderate garlic flavor | None noted |
| 0.01 | Stronger, spicier | None noted |
| 0.025 | Similar to 0.01%, possibly stronger | None noted |
| 0.05 | Stronger than 0.025% | None noted |
| 0.1 | Not as spicy, “overcooked” flavor, | Thinner, darker in color |
| | becomes too sweet | |

White Cranberry Apple Juice (Ocean Spray ®)

| | | |
|---|---|---|
| None | Mild, slightly tart, apple flavor | None noted |
| 0.01 | Less tart, increased flavor, more | None noted |
| | mellow | |
| 0.025 | Stronger flavor | None noted |
| 0.05 | Increased flavor, sweet aftertaste | None noted |
| 0.1 | Strongest flavor (apple) | None noted |

Chunky Garden Mushroom and Green Pepper Pasta Sauce (Prego ®)

| | | |
|---|---|---|
| None | Zesty sauce but not too spicy; taste | None noted |
| | of green peppers in sauce | |
| 0.01 | Smoother sauce, less mouth feel, less | None noted |
| | green pepper taste, still zesty | |
| 0.025 | Smoother sauce, less mouth feel, less | None noted |
| | green pepper taste, still zesty | |
| 0.05 | Reduced mouth feel and spiciness; | None noted |
| | “overcooked” | |
| 0.1 | Decreased spiciness, overcooked | None noted |
| | taste | |

Low Calorie Apple Cranberry Juice (Welch's ®)

| | | |
|---|---|---|
| None | Apple flavor, sweetener aftertaste | None noted |
| 0.01 | Off note (like green apples), | None noted |
| | decreased apple flavor, aftertaste | |
| 0.025 | Off note, sweetener aftertaste | None noted |
| 0.05 | Similar taste to 0.01 and 0.025 | Darker than control, |
| | | cloudy |
| 0.1 | Similar taste to 0.01 and 0.025 | Darker than control, |
| | | cloudy |

Light Cranberry Juice (Dole ®)

| | | |
|---|---|---|
| None | Tart, moderate cranberry flavor, | None noted |
| | some aftertaste but still drinkable | |
| 0.01 | Tarter, increased cranberry flavor, | None noted |
| | aftertaste there but less noticeable | |
| 0.025 | Sweeter, less cranberry, sweetener | None noted |
| | more prominent | |
| 0.05 | Tarter and not as sweet as 0.025%, | None noted |
| | less artificial sweetener taste, | |
| | increased cranberry flavor | |
| 0.1 | Increased tartness and cranberry | None noted |

### Example 11

### Evaluation of Taste and Color Loss After Enzymatic Treatment of Fruit Juices

The purpose of this example was to evaluate the taste of and color loss from colored fruit juices after they were treated with Protease M. In general, 25 mL aliquots of a fruit juice (cranberry juice, cranberry and apple juice mixture, cranberry juice and tea mixture, and grape juice) were transferred via pipette into tared sterile centrifuge tubes. The procedure was performed three (3) times for each dose level as described below.

Aliquots of (a) Protease M (100 mg/mL), (b) β-glycosidase (100 mg/mL), or (c) nothing (control) were added to each tube, where the concentrations of each enzyme solution was 0.01, 0.025, 0.05, 0.075, and 0.1% w/v. Each tube was shaken to mix, then incubated at 50° C. and 165 RPM for 3 hr.

The enzymes were then inactivated by heating the beverage products to 70° C. for 1 hr. Each sample was centrifuged at 9700 rpm for 10 minutes. The supernatant was poured off, and the tube was carefully dried and weighed to determine the weight of precipitated colored material.

The weight of the dried precipitate from each sample was used to evaluate the extent of color loss from each sample of fruit juice. The data was analyzed using SigmaStat® Software (Systat Software, Inc., Point Richmond, Calif.). The supernatant from each sample was taste-tested according to the procedure described above.

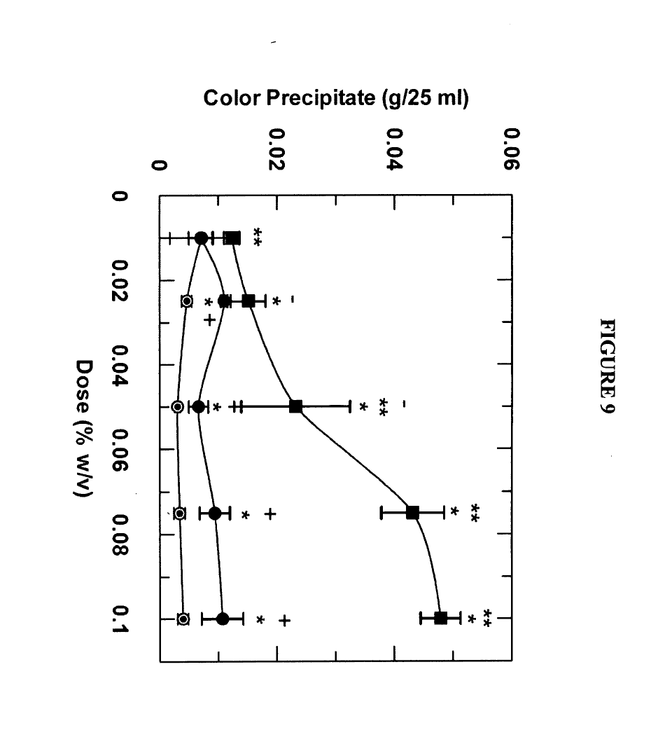

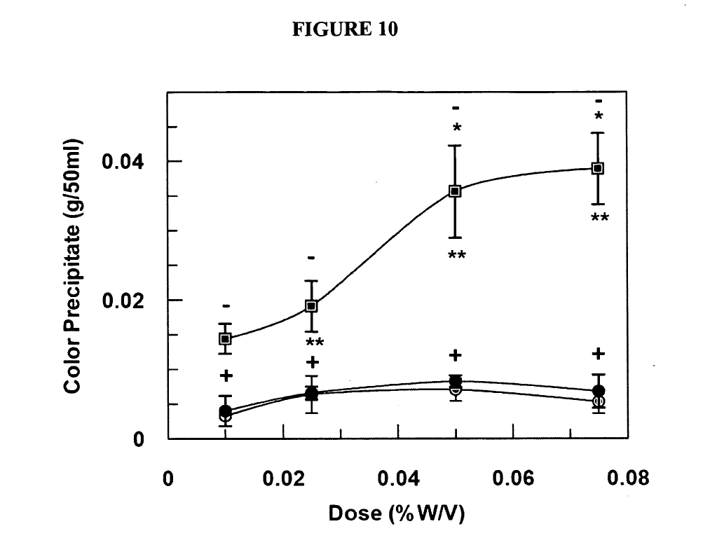

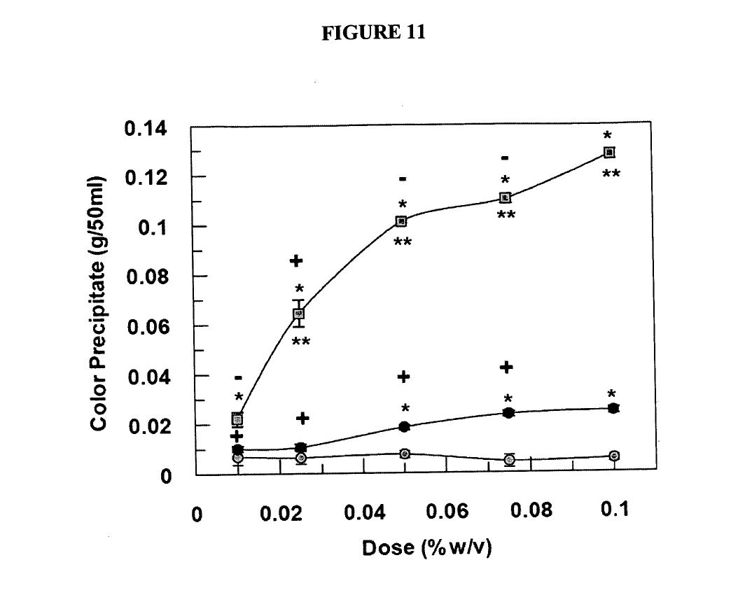

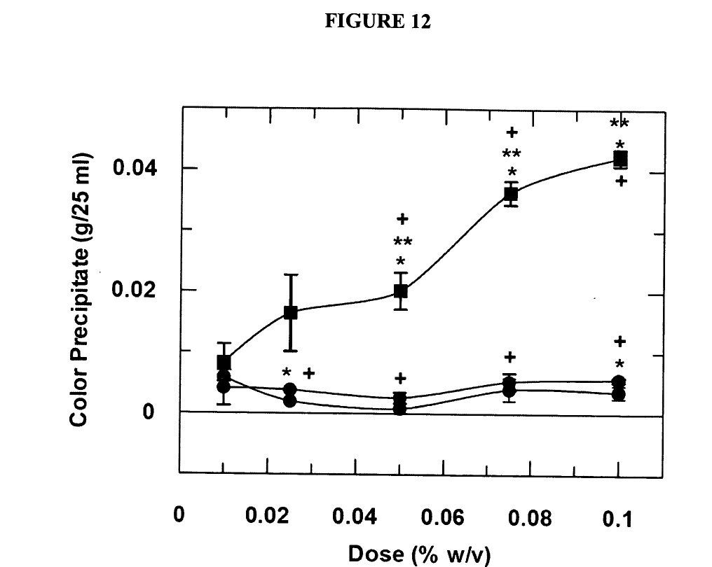

Each of FIGS. 9-12 present color loss and taste data for the three samples for each kind of fruit juice. Fruit juices that were treated with Protease M mixture resulted in the least color loss but the greatest enhancement in taste relative to the control and those fruit juice samples treated with β-glycosidase.

The results in Table 4 above and those presented in FIG. 9-12 indicate that cranberry juice is often perceived as being sweeter when it is treated with an enzyme composition comprising Protease M. Although not wishing to be bound by any certain theory, it is believed that the Protease M composition unmasks sweet flavors in bitter fruit juices such as cranberry juice to give the overall effect of a sweeter juice. The masked sweet flavor is believed to arise from either natural sugars or those added to the juice. This is important because less sugar can be added to juices that are treated according to the invention, while the juices maintain the same level of sweetness.

The invention has been disclosed broadly and illustrated in reference to representative embodiments described above. Those skilled in the art will recognize that various modifications can be made to the present invention without departing from the spirit and scope thereof.

We claim:

1. A food or beverage composition comprising (i) a food or beverage comprising a glycone and (ii) an enzyme composition having an enzyme activity profile that comprises glucosidase activity, β-glycosidase activity, protease activity, lipase activity, amylase activity, glucoamylase activity, xylanase activity, and pectinase activity, wherein the food or beverage composition exhibits an increased aglycone content and/or enhanced flavor relative to a corresponding composition that does not comprise the enzyme composition.

2. The composition according to claim 1, wherein the enzyme activity profile comprises one or more of:
a glucosidase activity of about 40 to about 70 u/g;
a β-glycosidase activity of about 0.3 to about 0.9 u/g;
a protease activity of about 4,000 to about 8,000 u/g;
a lipase activity of about 300 to about 500 u/g;
an amylase activity of about 160,000 to about 190,000 u/g;
a glucoamylase activity of about 24,000 to about 28,000 u/g;
a xylanase activity of about 11,000 to about 14,000 u/g; and
a pectinase activity of about 40 to about 120 u/g.

3. The composition of claim 1, wherein the enzyme composition comprises one or more enzymes selected from the group consisting of glucosidase, β-glycosidase, protease, lipase, amylase, glucoamylase, xylanase, and pectinase.

4. The composition of claim 1, wherein the enzyme composition comprises Protease M.

5. The composition of claim 1, wherein the beverage composition is selected from the group consisting of fruit juice, tea, alcoholic beverage and combinations thereof.

6. The composition of claim 5, wherein the beverage composition is a fruit juice, and the fruit juice is selected from the group consisting of cranberry, cherry, apple, tomato, orange, grapefruit, raspberry, and combinations thereof.

7. A food or beverage composition prepared by a process comprising contacting a food or beverage comprising a glycone with an enzyme composition having an enzyme activity profile that comprises glucosidase activity, β-glycosidase activity, protease activity, lipase activity, amylase activity, glucoamylase activity, xylanase activity, and pectinase activity, and wherein the food or beverage composition exhibits an increased aglycone content and/or enhanced flavor relative to a corresponding product that does not comprise the enzyme composition.

8. The process of claim 7, wherein the enzyme composition comprises one or more enzymes selected from the group consisting of glucosidase, β-glycosidase, protease, lipase, amylase, glucoamylase, xylanase, and pectinase.

9. The process of claim 7, wherein the enzyme composition comprises Protease M.

10. A method of enhancing the flavor of a food or beverage comprising contacting a food or beverage with an enzyme composition having an enzyme activity profile that comprises glucosidase activity, β-glycosidase activity, protease activity, lipase activity, amylase activity, glucoamylase activity, xylanase activity, and pectinase activity.

11. The method of claim 10, wherein the enzyme composition comprises one or more enzymes selected from the group consisting of glucosidase, β-glycosidase, protease, lipase, amylase, glucoamylase, xylanase, and pectinase.

12. The method of claim 10, wherein the enzyme composition comprises Protease M.

13. A method of increasing the aglycone content of a food or beverage comprising contacting a food or beverage comprising a glycone with an enzyme composition having an enzyme activity profile that comprises glucosidase activity, β-glycosidase activity, protease activity, lipase activity, amylase activity, glucoamylase activity, xylanase activity, and pectinase activity.

14. The method of claim 13, wherein the enzyme composition comprises one or more enzymes selected from the group consisting of glucosidase, β-glycosidase, protease, lipase, amylase, glucoamylase, xylanase, and pectinase.

15. The method of claim 13, wherein the enzyme composition comprises Protease M.

16. An enzyme composition comprising glutaminase, β-glycosidase, protease, lipase, amylase, glucoamylase, xylanase, and pectinase.

17. The composition according to claim 16, wherein the composition further comprises one or more buffers.

18. The composition according to claim 16, wherein the composition further comprises one or more of enzyme RP-1, deaminase and glutaminase.

19. The composition according to claim 16, having an enzyme activity profile comprising one or more of:
a β-glycosidase activity of about 0.3 to about 0.9 u/g;
a protease activity of about 4,000 to about 8,000 u/g;
a lipase activity of about 300 to about 500 u/g;
an amylase activity of about 160,000 to about 190,000 u/g;
a glucoamylase activity of about 24,000 to about 28,000 u/g;
a xylanase activity of about 11,000 to about 14,000 u/g; and
a pectinase activity of about 40 to about 120 u/g.

20. A food or beverage product, wherein the product comprises a flavor-enhancing amount of an enzyme composition having an enzyme activity profile comprising β-glycosidase activity, protease activity, lipase activity, amylase activity, glucoamylase activity, xylanase activity, and pectinase activity.

21. The product according to claim 20, wherein the enzyme activity profile further comprises glutaminase activity.

22. The product of claim 20, wherein the enzyme composition comprises one or more enzymes selected from the group consisting of β-glycosidase, protease, lipase, amylase, glucoamylase, xylanase, and pectinase.

23. The product of claim 20, wherein the enzyme composition comprises Protease M.

24. A process for producing a food or beverage product having an enhanced flavor profile, comprising the step of contacting the food or beverage product with a flavor-enhancing amount of an enzyme composition having an enzyme activity profile comprising β-glycosidase activity, protease activity, lipase activity, amylase activity, glucoamylase activity, xylanase activity, and pectinase activity, whereby the flavor profile of the food or beverage product is enhanced.

25. The process according to claim 24, wherein the process further comprises, after said contacting step, the step of heating the food or beverage product for a time and at a temperature sufficient to inactivate said enzyme composition.

26. The process of claim 24, wherein the enzyme composition comprises one or more enzymes selected from the group consisting of β-glycosidase, protease, lipase, amylase, glucoamylase, xylanase, and pectinase.

27. The process of claim 24, wherein the enzyme composition comprises Protease M.

28. A food or beverage product obtained by the process according to claim 24.
